(19) United States
(12) Patent Application Publication
(10) Pub. No.: US 2023/0191134 A1
(43) Pub. Date: Jun. 22, 2023

(54) IMPLANTABLE MEDICAL DEVICE WITH SYSTEM INTEGRITY DETERMINATION FOR EXPEDITED PATIENT DISCHARGE

(71) Applicant: Medtronic, Inc., Minneapolis, MN (US)

(72) Inventors: Eric A. Schilling, Ham Lake, MN (US); Elizabeth A. Mattson, Eagan, MN (US)

(21) Appl. No.: 18/062,332

(22) Filed: Dec. 6, 2022

Related U.S. Application Data
(60) Provisional application No. 63/265,550, filed on Dec. 16, 2021.

(51) Int. Cl. A61N 1/37; A61B 5/00

(52) U.S. Cl. CPC A61N 1/3702; A61B 5/0031

(57) ABSTRACT

An implantable medical device (IMD) performs, within a first predetermined time following an implantation, a first device test sequence over an evaluation period. The device test sequence includes at least two of: detecting an impedance for at least one electrical path having at least one electrode, and comparing the impedance to a first predetermined impedance threshold to determine a connection status of the IMD; comparing, over an electrogram (EGM) test period, at least one EGM event of the patient against a first predetermined EGM event threshold; determining a first pacing capture threshold of the IMD; and detecting at least one clinical or patient-specific physiologic metric, and comparing the physiologic metric to a first predetermined physiologic metric threshold. The IMD transmits within a second predetermined time a status signal to an external device indicating a status of at least one of the diagnostic tests in the first device test sequence.

This application claims the benefit of U.S. Provisional Patent Application Ser. No. 63/265,550, filed Dec. 16, 2021, the entire content of which is incorporated herein by reference.

### BACKGROUND

In some examples, implantable medical devices (IMD), such as, for example, cardiac pacemakers, include leads with a lead body containing one or more elongated electrical conductors. The electrical conductors extend through the lead body from a connector assembly provided at a first lead end proximal a housing of an associated IMD to one or more electrodes located at the distal lead end or elsewhere along the length of the lead body. The conductors connect stimulation and/or sensing circuitry within the IMD housing to respective electrodes or sensors. Each electrical conductor is typically electrically isolated from other electrical conductors and is encased within an outer sheath that electrically insulates the lead conductors from body tissue and fluids.

Therapeutic electrical stimulation provided by the leads connected to the IMD may include signals such as pulses or shocks for pacing, cardioversion or defibrillation. In some cases, a medical device may sense intrinsic depolarizations of the heart, and control delivery of stimulation signals to the heart based on the sensed depolarizations. An IMD may also be used to conduct temporary cardiac pacing following the end of an operative procedure, or may be implanted on a temporary basis (for example, for up to about 90 days) to provide pacing support to patients who may have temporary conduction disturbances, or as a bridge between permanent implants in cases of device or system infection.

In some examples, the implantation of an IMD, such as a temporary or permanent pacemaker, can utilize one or more custom support instruments such as, for example, a device programmer, to perform a number of status checks on the medical device at the time of implant. These confirmation checks help to ensure that the medical device is properly implanted and operational post implant procedure, and can be important to ensure that the system provides prescribed therapy to a patient beginning at the time of implant.

### SUMMARY

An implantable medical device (IMD) may be configured to automatically initiate a device test sequence including one or more qualification tests. For example, the IMD may be programmed to initiate the qualification tests on the day the patient is discharged from the hospital, beginning when a clock in the IMD crosses midnight (12:00 AM). For example, the IMD may perform a qualification test such as a pacing capture threshold (PCT) at about 1:00 AM on the first day after the implant is complete, or may perform a lead implant impedance check at about 3:00 AM on the first day post-implant. The IMD may also be configured to initiate a qualification test based on one or more patient cardiac events sensed by the IMD such as, for example, P-wave amplitude or R-wave amplitude, at about 2:15 AM on the first day post-implant. After these device qualification tests are performed, the patient can manually submit or initiate, the morning of the day after discharge, a status update to a remote monitoring system such as the system available from Medtronic, Inc., Minneapolis, Minn., under the trade designation CARELINK™.

To reduce potential patient exposure to pathogens such as methicillin-resistant Staphylococcus aureus (MRSA), or viruses such as COVID-19, and to reduce overall procedure costs, in some cases it may be desirable to discharge a patient following an IMD implant procedure on a more expedited basis than has been customary, e.g., on the same day in which an IMD implant is performed. To ensure that the IMD implant procedure is successful, the device test sequence performed by the IMD should produce results within a clinically appropriate range of values over an initial evaluation period. However, some of the qualification tests performed by the IMD may not be successful on a single attempt. For example, a PCT qualification test can abort or be deemed a “fail” if cardiac rhythm is unstable (for example, upon detection of premature ventricular contractions (PVC)). In another example, a qualification test based on one or more sensed cardiac events, such as R-wave measurements, can be aborted if the IMD is not in a 100% pacing mode at the time of measurement. In these cases, the data available to the clinician at the time of the manual transmission uplink by the patient to the remote monitoring system may include incomplete or inaccurate results for some of the clinically useful measurements, which can lead to uncertainty about the overall status of the IMD or a lead attached to the IMD.

In some examples, in the techniques of the present disclosure the IMD is configured to automatically perform clinically necessary device qualification tests to determine, in appropriate cases, the possibility of a same-day patient discharge. In some examples, in the techniques of the present disclosure the IMD automatically initiates a device test sequence within a predetermined time following completion of the implant procedure (e.g., within 1-8 hours following the procedure), while the patient remains in the hospital and under observation by hospital staff, and performs the device test sequence multiple times over shorter intervals than is conventional. This accelerated schedule accumulates a larger volume of clinical data regarding the success or failure of the implant procedure that may be used by a clinician to make an informed discharge decision.

After the patient is discharged from the hospital, these clinical data are also available when the patient manually transmits an implant confirmation signal to the remote monitoring system. The larger and more complete collection of data can provide the clinician with a more accurate picture of implant performance, and increase confidence in the implant procedure. In some examples, the IMD may be configured to automatically transmit an implant confirmation signal to the remote monitoring system.

After the manual patient uplink, or the automatic uplink from the IMD, the IMD may be configured to initiate a second device test sequence that is different from the initial device test sequence performed by the IMD following completion of the implantation procedure. In some examples, the second device test sequence may include different parameters that are more stringent than the initial set of parameters utilized in the initial device test sequence on the day of implant.

In some examples, the device test sequence may be initiated by the IMD within hours of implant and repeatedly performed by the IMD until the IMD detects an intention to discharge the patient. In one example, if the IMD includes an accelerometer, the IMD may detect that the patient is vertical and out of bed for a period of time, or is sufficiently ambulatory, to indicate that patient discharge may be appropriate. In another example, the IMD may detect a patient location based on information obtained from a mobile device associated with the patient.

In another example, the device test sequence may be initiated by the IMD within hours of implant and repeatedly performed by the IMD, and the IMD may provide a summary of one or more of the qualification tests in the device test sequence to an external device such as, for example, a mobile device, a device programmer, or the like, prior to discharge of the patient. In some cases, the summary may render unnecessary a full device interrogation of the IMD by the device programmer. In one example, the IMD may transmit a summary of the qualification test data to a mobile device in Bluetooth low energy (BLE) advertising packets. Following the BLE transmission, the IMD may be configured to automatically transmit an implant confirmation signal to the remote monitoring system. After the automatic uplink from the IMD, the IMD may be configured to initiate a second device test sequence that is different from the initial device test sequence performed by the IMD following completion of the implantation procedure.

The IMD may automatically perform the device test sequences without requiring any external instrument or programming device, as such the method of the present disclosure can simplify the IMD implantation procedure and evaluation of device integrity and performance. In addition, the data collected by the IMD can allow a clinician to confidently discharge a patient from the hospital more quickly, even on the day of implant, which can reduce procedure costs and limit potential patient exposure to pathogens.

In one aspect, the present disclosure is directed to a method, including: initiating, by an implantable medical device (IMD), within a first predetermined time following an implantation of the IMD in a patient, a first device test sequence in which the IMD performs at least two of the following qualification tests over an evaluation period: (1) detecting an impedance for at least one electrical path that includes at least one electrode, and comparing the impedance to a first predetermined impedance threshold to determine a connection status of the IMD to the at least one electrode; (2) comparing, over an electrogram (EGM) test period, at least one EGM event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metric threshold; and transmitting, by the IMD, to an external device, within a second predetermined time, a status signal indicating a status of at least one of the diagnostic tests in the first device test sequence.

In another aspect, the present disclosure is directed to a method, including: initiating, by an implantable medical device (IMD), within a first predetermined time following an implantation of the IMD in a patient, a first device test sequence in which the IMD performs at least one of the following qualification tests over an evaluation period: (1) detecting an impedance for at least one electrical path that includes at least one electrode, and comparing the impedance to a first predetermined impedance threshold to determine a connection status of the IMD to the at least one electrode; and (2) comparing, over an EGM test period, at least one electrogram (EGM) event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metrics threshold; and transmitting by the IMD, to an external device, within a second predetermined time a status signal indicating a status of at least one of the diagnostic tests in the first device test sequence.

In another aspect, the present disclosure is directed to a method, including: initiating, by an implantable medical device (IMD) with an accelerometer, within a first predetermined time following an implantation of the IMD in a patient, a first device test sequence in which the IMD performs at least two of the following qualification tests over an evaluation period: (1) detecting an impedance, and comparing the impedance to a first predetermined impedance threshold to determine a connection status of the IMD to at least one electrode; and (2) comparing, over an EGM test period, at least one electrogram (EGM) event of the patient against a first predetermined EGM event threshold; and (3) determining a first pacing capture threshold (PCT) of the IMD; and (4) detecting at least one clinical or patient-specific physiologic metric, and comparing the physiologic metric to a first predetermined physiologic metrics threshold; and transmitting by the IMD, to an external device, within a second predetermined time period, a status signal indicating a status of at least one of the diagnostic tests in the first device test sequence; and determining, by the IMD, a discharge status of the patient by at least one of: determining, by the accelerometer in the IMD, at least one of a body posture or an ambulation of the patient, and comparing the at least one of body posture or ambulation of the patient against a threshold for discharge; or determining, by the IMD, a patient location.

In another aspect, the present disclosure is directed to method, including: initiating, by an implantable medical device (IMD), within a first predetermined time following an implantation of the IMD in a patient, a first device test sequence in which the IMD performs at least one of the following qualification tests over an evaluation period: (1) detecting an impedance for at least one electrical path that includes at least one electrode, and comparing the impedance to a first predetermined impedance threshold to determine a connection status of the IMD to the at least one electrode; and (2) comparing, over an EGM test period, at least one electrogram (EGM) event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metrics threshold; and transmitting by the IMD, to an external device, within a second predetermined time period a status signal indicating a status of at least one of the diagnostic tests in the first device test sequence, wherein the first status signal is chosen from complete/pass, complete/fail, and incomplete.

In yet another aspect, the present disclosure is directed to a system including: at least one implantable medical lead having one or more electrodes; an implantable medical device (IMD) coupled to the at least one lead, wherein the at least one lead senses a cardiac electrogram (EMG) signal of a patient via the electrodes; and wherein the IMD includes a processor that causes the IMD to initiate, following implantation of the IMD, a device test sequence in which the IMD performs any of the methods above.

The details of one or more examples are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a conceptual drawing illustrating an example system that includes a temporary or permanent implantable medical device (IMD) coupled to implantable medical leads.

FIG. 2 is a functional block diagram illustrating an example configuration of the IMD of FIG. 1.

FIGS. 3-6 are flow diagrams of examples of device test sequences according to the present disclosure.

Like symbols in the drawings indicate like elements.

### DETAILED DESCRIPTION

In general, in this application an implantable medical device (IMD) is configured to initiate a device test sequence including a number of qualification tests within a predefined period of time (e.g., no more than about 1 hour, or about 1 hour to about 8 hours, ±0.5 hours) following initial implantation of the IMD. In some examples, the device test sequence may be used to evaluate the readiness to discharge a patient from the hospital or other facility where the implantation procedure was performed. The IMD performs the device test sequence periodically over an evaluation period, and provides a periodic status signal for at least some of the qualification tests.

These qualification tests are performed automatically by the IMD, which in this application means that the IMD performs the tests or sequence of tests without input from an external programmer or other monitoring device, although in some examples the IMD may perform the qualification test or sequence of tests following interrogation by, or instructions from, an external programmer. In some examples, the device test sequence initiated by the IMD includes one or more of the following qualification tests: (1) periodic lead impedance monitoring to, for example, detect connection to electrodes, (2) measurement by the IMD of sensed electrogram (EGM) events in the heart of the patient including one or more of P-wave amplitudes, R-wave amplitudes, and COI parameters; (3) determination of pacing capture threshold (PCT) of the implanted device; and (4) measurement of selected patient physiologic metrics. The IMD may optionally include an accelerometer suitable for detecting that the patient is vertical and out of bed for a period of time, or is sufficiently ambulatory, to indicate that patient discharge may be appropriate. The IMD may optionally detect a patient location based on information obtained from a mobile device associated with the patient. The IMD produces an amount of clinical data sufficient to allow a clinician to determine more rapidly the success (or failure) of the implantation procedure, which may allow patient discharge from a hospital on the day of implantation, or require that the patient remain in the hospital for a more extended time period.

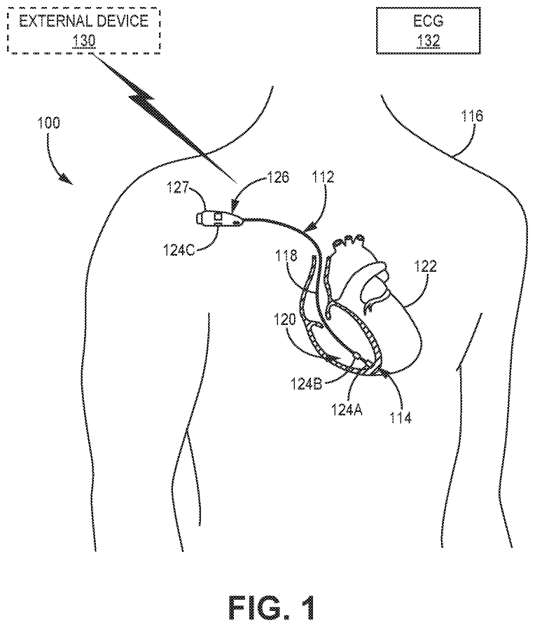

FIG. 1 is a conceptual diagram illustrating a portion of an example implantable medical device system 100 in accordance with one or more aspects of this disclosure. Implantable medical device system 100 may function as a single chamber, e.g., ventricular, pacemaker, as illustrated by the example of FIG. 1, or as dual-chamber pacemaker that delivers pacing to a heart 122 of patient 116. While FIG. 1 shows the medical device system 100 implanted on the right side of the body of the patient 116, it should be understood that the system 100 can be implanted at any location in the body of the patient 116, or may even be a temporary system 100 functioning outside the body of the patient 116. In addition, the shape and configuration of the system 100 shown in FIG. 1 is merely conceptual, and is not intended to be limiting.

In the example of FIG. 1, implantable medical device system 100 includes one or more implantable medical leads 112 and an implantable medical device (IMD) 126. However, the methods of the present disclosure are not limited to IMDs including leads, and may also be employed with leadless pacemakers such as, for example, those available from Medtronic, Inc., under the trade designation MICRA™. In some examples, the techniques of the present disclosure may be implemented in IMDs other than pacemakers, such as implantable monitors or neurostimulators.

In the embodiment of FIG. 1, the implantable medical lead 112 includes an elongated lead body 118 with a distal portion 120. Distal portion 120 of implantable medical lead 112 is positioned at a target site 114 within a heart 122 of a patient 116. Distal portion 120 may include one or more electrodes. Target site 114 may be located at a wall of a ventricle of heart 122. In various examples, the lead 112 may be a unipolar, a bipolar, or a multipolar lead. The IMD 126 may additionally or alternatively include one or more optical sensors, accelerometers, temperature sensors, chemical sensors, light sensors, pressure sensors, in some examples. Such sensors may detect one or more physiological parameters indicative of a patient condition.

A clinician may maneuver distal portion 120 through the vasculature of patient 116 to position distal portion 120 at or near target site 114. For example, the clinician may guide distal portion 120 through the superior vena cava (SVC) to target site 114 on or in a ventricular wall of heart 122, e.g., at the apex of the right ventricle as illustrated in FIG. 1. In some examples, other pathways or techniques may be used to guide distal portions 120 into other target implant sites within the body of patient 116. Implantable medical device system 100 may include a delivery catheter and/or outer member (not shown), and implantable medical lead 112 may be guided and/or maneuvered within a lumen of the delivery catheter in order to approach target site 114.

Implantable medical lead 112 may include electrodes 124A and 124B (collectively, “electrodes 124”) configured to be positioned on, within, or near cardiac tissue at or near target site 114. In some examples, electrodes 124 are configured to function as electrodes to, for example, provide pacing to heart 122. Electrodes 124 may be electrically connected to conductors (not shown) extending through lead body 118. In some examples, the conductors are electrically connected to therapy delivery circuitry of IMD 126, with the therapy delivery circuitry configured to provide electrical signals through the conductor to electrodes 124. Electrodes 124 may conduct the electrical signals to the target tissue of heart 122, causing the cardiac muscle, e.g., of the ventricles, to depolarize and, in turn, contract at a regular interval. Electrodes 124 may also be connected to sensing circuitry of IMD 126 via the conductors, and the sensing circuitry may sense activity of heart 122 via electrodes 124. Electrodes 124 may have various shapes such as tines, helices, screws, rings, and so on. Again, although a bipolar configuration of lead 112 including two electrodes 124 is illustrated in FIG. 1, in other examples IMD 126 may be coupled to leads including different numbers of electrodes, such as one electrode, three electrodes, or four electrodes. In other examples (not shown in FIG. 1), the IMD 126 can be connected to two leads (atrium and right ventricle) or three leads (A, RV, LV), or other electrode or lead configurations.

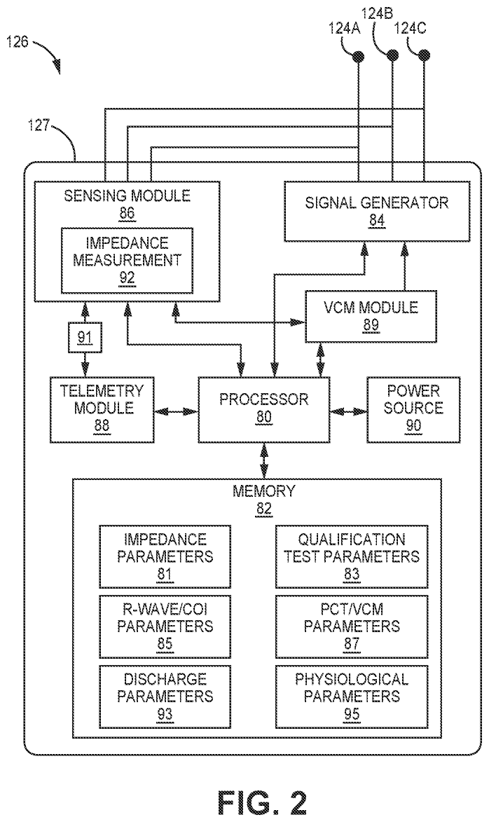

In some examples, as illustrated schematically in FIG. 1, the IMD 126 includes one or more housing electrodes 124C, which may be formed integrally with an outer surface of a hermetically-sealed housing 127 of the IMD 126 or otherwise coupled to the housing 127. In some examples, the housing electrode 124C is defined by an uninsulated portion of an outward facing portion of the housing 127 of the IMD 126. Other divisions between insulated and uninsulated portions of the housing 127 may be employed to define two or more housing electrodes. In some examples, the housing electrode 124C can include substantially all of the housing 127. Any of the electrodes 124A, 124B may be used for unipolar sensing or pacing in combination with the housing electrode 124C. As described in further detail with reference to FIG. 2, the housing 127 may enclose a stimulation signal generator that generates cardiac pacing pulses and defibrillation or cardioversion shocks, as well as a sensing module for monitoring the patient's heart rhythm.

The configuration of the therapy system 100 illustrated in FIG. 1 is merely one example. In other examples, a therapy system may include epicardial leads, subcutaneous, substernal, and/or patch electrodes instead of or in addition to the transvenous leads 118 illustrated in FIG. 1. Further, the IMD 126 need not be implanted within the patient 116. In examples in which the 1 MB 126 is not implanted in the patient 116, the 1 MB 12 may deliver therapies to the heart 122 via percutaneous leads that extend through the skin of patient 116 to a variety of positions within or outside of heart 122.

In one or more examples, IMD 126 includes electronic circuitry contained within an enclosure where the circuitry may be configured to deliver cardiac pacing. In the example of FIG. 1, the electronic circuitry within IMD 126 may include therapy delivery circuitry electrically coupled to electrodes 124. The electronic circuitry within IMD 126 may also include sensing circuitry configured to sense electrical activity of heart 122 via electrodes 124. The therapy delivery circuitry may be configured to administer cardiac pacing via electrodes 124, e.g., by delivering pacing pulses in response to expiration of a timer and/or in response to detection of the activity (or absence thereof) of the heart.

In some examples, the system 100 includes an optional programmer 130. For example, optional programmer 130 can be a handheld computing device such as a tablet or a phone, a computer workstation, or a networked computing device. The optional programmer 130 can include a user interface that receives input from a clinician, which can include a keypad and a suitable display such as, for example, a touch screen display, or a peripheral pointing device, such as a mouse, via which a user may interact with the user interface. The clinician may also interact with the programmer 130 remotely via a networked computing device.

The programmer 130 may be a hand-held computing device with a display viewable by the user and an interface for providing input to programmer 130 (i.e., a user input mechanism). For example, the programmer 130 may include a small display screen (e.g., a liquid crystal display (LCD) or a light emitting diode (LED) display) that presents information to the user. In addition, the programmer 130 may include a touch screen display, keypad, buttons, a peripheral pointing device, voice activation, or another input mechanism that allows the user to navigate through the user interface of programmer 130 and provide input. If programmer 130 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, programmer 130 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When programmer 130 is configured for use by the clinician, programmer 130 may be used to transmit instructions to 1 MB 126 and to receive measurements. Example instructions may include requests to set electrode combinations for sensing and any other information that may be useful for programming into IMD 126. The clinician may also configure and store operational parameters for IMD 126 within the 1 MB 126 with the aid of programmer 130. In some examples, programmer 130 assists the clinician in the configuration of IMD 126 by providing a system for identifying potentially beneficial operational parameter values.

Whether programmer 130 is configured for clinician or patient use, programmer 130 is configured to communicate with IMD 126 and, optionally, another computing device (not illustrated in FIG. 1), via wireless communication. The programmer 130, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, the IMD 126 may signal the programmer 130 to further communicate with and pass the alert through a network such as those available under the trade designation Medtronic CARELINK Network from Medtronic, Inc., of Minneapolis, Minn., or some other network linking the patient 116 to a clinician.

In some examples, the system 100 includes an optional electrocardiogram (ECG) monitor 132. In some methods of the present disclosure, if the programmer 130 is not available, the EGC monitor 132 can provide a clinician with feedback regarding the rhythm and electrical activity of the heart following an IMD implantation procedure. In various examples, the ECG monitor can include one or more leads (not shown in FIG. 1), may include surface sensors attached to the skin of the patient (not shown in FIG. 1), or may be a wireless monitor without leads.

FIG. 2 is a functional block diagram illustrating an example configuration of an example IMD such as the IMD 126 described in FIG. 1. In the illustrated example, the IMD 126 includes a processor 80, a memory 82, a signal generator 84, a sensing module 86, a telemetry module 88, an optional accelerometer 87, a ventricular capture management (VCM) module 89, and power source 90. The memory 82 includes computer-readable instructions that, when executed by the processor 80, cause the IMD 126 and the processor 80 to perform various functions described herein. The memory 82 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital or analog media.

The processor 80 may include any one or more of a microprocessor, a controller, a digital signal processor (DSP), an application specific integrated circuit (ASIC), a field-programmable gate array (FPGA), or equivalent discrete or analog logic circuitry. In some examples, the processor 80 may include multiple components, such as any combination of one or more microprocessors, one or more controllers, one or more DSPs, one or more ASICs, or one or more FPGAs, as well as other discrete or integrated logic circuitry. The functions attributed to the processor 80 herein may be embodied as software, firmware, hardware or any combination thereof.

The processor 80 controls the signal generator 84 to deliver stimulation therapy to the heart 122 according to a selected one or more of therapy programs, which may be stored in the memory 82. For example, the processor 80 may control the signal generator 84 to deliver electrical pulses with the amplitudes, pulse widths, frequency, or electrode polarities specified by the selected one or more therapy programs. As discussed in more detail below, the processor 80 may control the signal generator to pace the heart in a number of different modes including, but not limited to, VOO, VVI, and OVO.

The signal generator 84 is electrically coupled to the electrodes 124A, 124B, e.g., via conductors of the respective leads 120, or, in the case of the housing electrode 124C, via an electrical conductor disposed within the housing 127 of the 1 MB 126. The signal generator 84 generates and delivers electrical stimulation therapy to the heart 122. For example, the signal generator 84 delivers pacing, cardioversion, or defibrillation therapy in the form of electrical pulses via the electrodes 124A-C. In other examples, the signal generator 84 may deliver one or more of these types of therapy in the form of other signals, such as sine waves, square waves, or other substantially continuous time signals.

The signal generator 84 may include circuitry configured to generate such signals, such as one or more capacitors, charge pumps, current or voltage sources or regulators, transistors, or switches. The signal generator 84 may include a switch module, and the processor 80 may use the switch module to select, e.g., via a data/address bus, which of the available electrodes are used to deliver defibrillation pulses or pacing pulses and their polarity. The switch module may include a switch array, switch matrix, multiplexer, or any other type of switching device suitable to selectively couple stimulation energy to selected electrodes.

The electrical sensing module 86 monitors signals from at least one of the electrodes 124A-C to monitor electrical activity of the heart 122. The sensing module 86 may also include a switch module to select which of the available electrodes are used to sense the heart activity, depending upon which electrode combination is used in the current sensing configuration. In some examples, the processor 80 may select the electrodes that function as sense electrodes, i.e., select the sensing configuration, via the switch module within the sensing module 86. The processor 80 may control the functionality of the sensing module 86 by providing signals via a data/address bus.

The sensing module 86 may include one or more detection channels, each of which may include an amplifier. The detection channels may be used to sense the cardiac signals. Some detection channels may detect sensed cardiac events, such as, for example, electrogram signals such as R- or P-waves, or current of injury (COI) parameters, and provide indications of the occurrences of such events to the processor 80. In some examples, the COI parameters are based on the digitally converted signal.

For example, the sensing module 86 may include one or more channels each of which may include a sense-amplifier that compares the detected signal to a threshold. If the filtered and amplified signal is greater than the threshold, the channel indicates that a certain electrical cardiac event, e.g., depolarization, has occurred. The processor 80 then uses that detection in measuring frequencies of the sensed events. Different channels of the sensing module 86 may have distinct functions. For example, some various channels may be used to sense either atrial or ventricular events. In one example, at least one channel may include an R-wave amplifier that receives signals from the sensing configuration of the electrodes 124A-B, which are used for sensing and/or pacing in the right ventricle of the heart 122. In some examples, the R-wave amplifiers may take the form of an automatic gain controlled amplifier that provides an adjustable sensing threshold as a function of the measured R-wave amplitude.

In addition, in some examples, a channel may include a P-wave amplifier that receives signals from the electrodes 124A-B, which are used for pacing and sensing in the right atrium of the heart 122. In some examples, the P-wave amplifier may be an automatic gain controlled amplifier that provides an adjustable sensing threshold as a function of the measured P-wave amplitude of the heart rhythm. One or more of the sensing channels of sensing module 86 may also be selectively coupled to any of the electrodes 124A-C for unipolar sensing of R-waves or P-waves in any of chambers of the heart 122.

In some examples, signals from the selected sensing electrodes that are selected for coupling to this wide-band amplifier may be converted to multi-bit digital signals by an analog-to-digital converter provided by, for example, the sensing module 86 or the processor 80. The processor 80 may store signals the digitized versions of signals from the wide band channel in memory 82 as EGM signals. The storage of such EGMs in memory 82 may be under the control of a direct memory access circuit. In some examples, the processor 80 may employ digital signal analysis techniques to characterize the digitized signals from the wide band channel to, for example, detect R-waves, P-waves, COI parameters, or detect and classify the patient's heart rhythm.

If the IMD 126 generates and delivers pacing pulses to the heart 122, the processor 80 may include a pacer timing and control module, which may be embodied as hardware, firmware, software, or any combination thereof. The pacer timing and control module may comprise a dedicated hardware circuit, such as an ASIC, separate from other processor 80 components, such as a microprocessor, or a software module executed by a component of the processor 80, which may be a microprocessor or ASIC. The pacer timing and control module may include programmable counters that control the basic time intervals associated with VOO, OVO, DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber that is sensed, and the third letter may indicate the chamber in which the response to sensing is provided.

Intervals defined by the pacer timing and control module within the processor 80 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals to the sensing module 86 to blank one or more channels, e.g., amplifiers, for a period during and after delivery of electrical stimulation to the heart 122. The durations of these intervals may be determined by the processor 80 in response to stored data in the memory 82. The pacer timing and control module of the processor 80 may also determine the amplitude of the cardiac pacing pulses.

During pacing, escape interval counters within the pacer timing/control module of the processor 80 may be reset upon sensing of R-waves and P-waves with detection channels of the sensing module 86. Signal generator 84 may include pacer output circuits that are coupled, e.g., selectively by a switching module, to any combination of the electrodes 124A-C appropriate for delivery of a bipolar or unipolar pacing pulse to one of the chambers of the heart 122. The processor 80 may reset the escape interval counters upon the generation of pacing pulses by the signal generator 84, and thereby control the basic timing of cardiac pacing functions, including anti-tachyarrhythmia pacing.

The value of the count present in the escape interval counters when reset by sensed R-waves and P-waves may be used by the processor 80 to measure the durations of R-R intervals, P-P intervals, P-R intervals and R-P intervals, and these measurements may be stored in the memory 82.

The processor 80 may operate as an interrupt driven device, and is responsive to interrupts from the pacer timing and control module, where the interrupts may correspond to the occurrences of sensed P-waves and R-waves and the generation of cardiac pacing pulses. Any necessary mathematical calculations to be performed by the processor 80 and any updating of the values or intervals controlled by the pacer timing and control module of the processor 80 may take place following such interrupts. A portion of the memory 82 may be configured as a plurality of recirculating buffers, capable of holding series of measured intervals, which may be analyzed by the processor 80 in response to the occurrence of a pace or sense interrupt to determine whether the patient's heart 12 is presently exhibiting atrial or ventricular tachyarrhythmia, asystole, or the like.

In some examples, IMD 126 includes one or more accelerometers 91 (FIG. 2). The accelerometer 91 collects an accelerometer signal which reflects a measurement of a motion of the patient 116 (FIG. 1). In some cases, the accelerometer 91 may collect a three-axis accelerometer signal indicative of patient 116's movements within a three-dimensional Cartesian space. For example, the accelerometer 91 output signal may include a vertical axis accelerometer signal vector, a lateral axis accelerometer signal vector, and a frontal axis accelerometer signal vector. The vertical axis accelerometer signal vector may represent an acceleration of patient 116 along a vertical axis, the lateral axis accelerometer signal vector may represent an acceleration of patient 116 along a lateral axis, and the frontal axis accelerometer signal vector may represent an acceleration of patient 116 along a frontal axis. In some cases, the vertical axis substantially extends along a torso of patient 116 from a neck of patient 116 to a waist thereof, the lateral axis extends across a chest of patient 116 perpendicular to the vertical axis, and the frontal axis extends outward from and through the chest of patient 116, the frontal axis being perpendicular to the vertical axis and the lateral axis.

In some examples, processing circuitry 80 may be configured to identify, based on an accelerometer signal from the accelerometer 91 and discharge parameters 93 stored in the memory module 82, that the patient 116 is upright and out of bed following implantation of the IMD 126. In some examples, the processing circuitry 80 may be configured to identify, based on the signals from the accelerometer 91 and the discharge parameters 93, that the patient 116 is ambulatory. Either or both of the upright posture of the patient 116 and the ambulatory status of the patient 116 may be used to determine that the patient 116 is ready to be discharged from a hospital following implantation of the IMD 126. The comparison of the output signals of the accelerometer 91 to the discharge parameters 93 may be performed following the completion of the device test sequence performed by the IMD 126, or in parallel with the device test sequence.

In some examples, the discharge parameters 93 stored in the memory 82 may further optionally include location information for the patient 116 or of the hospital where the IMD 126 is implanted. In some examples, the IMD 126 may detect a location of the patient 116, and determine, based on the detected location information, a discharge status of the patient. For example, the IMD 126 may detect departure of the patient 116 from a particular area of the hospital or other clinical facility, detect that the patient 116 has been moved to a designated pre-discharge area of the hospital, or may detect that the patient 116 has left the hospital entirely.

In some examples, the memory 82 further includes a physiologic parameter module 95. The measurement module 86 of the IMD 126 may be configured to measure clinical or patient-specific physiologic parameters stored in the physiologic parameter module 95 such as, for example, heart rate, respiration rate, activity level, thoracic impedance, blood pressure, ejection fraction, heart sounds, arrythmia burden, percent pacing needed, and combinations thereof. The processor 80 may compare the measured physiological parameters to a predetermined physiological metrics threshold to provide patient status information or to provide data that can be used to evaluate discharge status for the patient.

The sensing module 86 and/or the processor 80 are configured to control the IMD 126 to initiate a device test sequence according to qualification test parameters 83 stored in memory 82. In some examples, the device test sequence(s) are initiated automatically (without further input from the programmer 130 or other device external to the IMD 126). In some examples, the device test sequence(s) are triggered after the one or more leads are connected to the IMD 126. In some examples, the device test sequence(s) are triggered upon activation of a leadless IMD 126, or attachment of leads to a temporary or a permanent external IMD 126.

The sensing module 86 and/or the processor 80 cause the IMD 126 to conduct, in series or in parallel, a device test sequence within a first predetermined time following the completion of the implantation procedure for the IMD 126. In some examples, the first predetermined time is about 0.5 hours to about 6 hours, or about 1 hour to about 3 hours, or about 1 hour to about 2 hours. In another example, the clinician can program an expected patient discharge time into the IMD 126. The expected patient discharge time may be entered into the IMD 126 at a particular time such as when the implant procedure begins or is complete. The IMD 126 may then initiate the device test sequence at a time prior to the expected patient discharge time, such that the device test sequence are complete and as current as possible at the time of patient discharge review.

The device test sequence performed by the IMD includes at least two of the following qualification tests:

(1) detecting an impedance for at least one electrical path that includes at least one electrode, and comparing the impedance to a first predetermined impedance threshold to determine a connection status of the IMD to the at least one electrode;

(2) comparing, over an EGM test period, at least one electrogram (EGM) event of the patient against a first predetermined EGM event threshold;

(3) determining a first pacing capture threshold (PCT) of the IMD; and

(4) measuring selected clinical or patient-specific physiological metrics and comparing the physiological metrics against a first predetermined physiologic metrics threshold.

In some examples, the IMD may optionally include an accelerometer suitable for detecting that the patient is vertical and out of bed for a period of time, or is sufficiently ambulatory, to indicate that patient discharge may be appropriate. The IMD may optionally detect a patient location based on information obtained from a mobile device associated with the patient.

In some examples, the IMD 126 performs the device test sequence over an evaluation period of about 12 hours to about 24 hours following completion of the implantation.

Once one or more of the qualification tests (1)-(4) return results in a passing range, in some examples the IMD automatically generates and sends to an external device such as a mobile phone, a tablet, a device programmer, a network such as Medtronic CARELINK and the like, a status signal providing a status of at least one of the diagnostic tests in the device test sequence. For example, the status signal generated by the IMD 126 may include an aggregate value indicating the success or failure of the implantation of the IMD 126, or a summary of one or more of the values of the qualification tests (1)-(4). In one example, the summary in the status signal may be limited to complete/pass, complete/fail, and incomplete. In various examples, the IMD 126 may generate the status signal either prior to or after the patient is discharged from the hospital. In another example, the status signal may include a comparison of any or all of the qualification test results against previously measured results and provided in a report to the clinician. The previously measured results could include manually initiated measurements performed at the time of implant, or prior automated tests. This would allow the clinician to see significant changes in the parameters since implant, or over time, as part of the decision making process for suitability to discharge the patient.

The IMD 126 generates the status signal within a second predetermined time following completion of the device test sequence. For example, the second predetermined time is at least once every 2 hours following implantation, or once every 3 hours, or once every 4 hours, or once every 6 hours, or any other predetermined period of time.

In some examples, the IMD generates no automatic status signal, but a clinician may review the results of the qualification tests performed by the IMD 126 and stored in the memory 82, and assess the success or failure of the implantation procedure.

In qualification test (1), the sensing module 86 and/or processor 80 are capable of collecting, measuring, and/or calculating impedance data according to impedance parameters 81 stored in the memory 82 for any of a variety of electrical paths that include two or more of the electrodes 124A-C. The impedance measurement module 92 in the IMD may measure impedance to determine proper lead connection, or can measure electrical parameter values during delivery of an electrical signal between at least two of the lead electrodes. The processor 80 may control signal generator 84 to deliver the electrical signal between the electrodes. The processor 80 may determine impedance values based on parameter values measured by the impedance measurement module 92, and store the measured impedance values in the memory 82.

In some examples, the processor 80 may perform an impedance measurement by controlling delivery, from the signal generator 84, of a voltage pulse between selected first and second electrodes. The impedance measurement module 92 may measure a resulting current, and the processor 80 may calculate a resistance based upon the voltage amplitude of the pulse and the measured amplitude of the resulting current. In other examples, the processor 80 may perform an impedance measurement by controlling delivery, from the signal generator 84, of a current pulse between first and second electrodes, the measurement module 92 may measure a resulting voltage, and the processor 80 may calculate a resistance based upon the current amplitude of the pulse and the measured amplitude of the resulting voltage. The measurement module 92 may include circuitry for measuring amplitudes of resulting currents or voltages, such as sample and hold circuitry.

In these examples, the signal generator 84 delivers signals that do not necessarily deliver stimulation therapy to the heart 122, due to, for example, the amplitudes of such signals and/or the timing of delivery of such signals. For example, these signals may include sub-threshold amplitude signals that may not stimulate the heart 122. In some cases, these signals may be delivered during a refractory period, in which case they also may not stimulate the heart 122. The IMD 126 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue of the heart 122.

In certain cases, the 1 MB 126 may measure impedance values that include both a resistive and a reactive (i.e., phase) component. In such cases, the 1 MB 126 may measure impedance during delivery of a sinusoidal or other time varying signal by the signal generator 84, for example. Thus, as used herein, the term “impedance” is used in a broad sense to indicate any collected, measured, and/or calculated value that may include one or both of resistive and reactive components.

The processor 80 may control a plurality of measurements of the impedance of any one or more electrical paths including combinations of the electrodes 124A-C according to the impedance parameters 81 stored therein. In some examples, the processor 80 determines that the qualification test (1) is a pass when processor 80 determines that the impedance measurement module 92 detects an impedance threshold stored in the memory 82. In some examples, an impedance measurement of about 300Ω to about 2000Ω, or about 300Ω to about 1000Ω, is sufficient to provide a pass for qualification test (1).

In some examples, before the IMD 126 performs the qualification test (1), the signal generator 84 paces the heart in VOO mode (ventricular pacing, no sensing). In some examples, the signal generator 84 paces the heart at about 80 beats per minute (bpm). In some examples, the signal generator 84 and the sensing module 86 may be used to pace the heart in VVI mode (sensed ventricular pacing) prior to the IMD performing the qualification test (1). For example, the signal generator 84 may pace the heart at about 60 bpm in response to detection of signals (e.g., detection of the absence of intrinsic R-waves) from the sensing module 86. In some examples, the heart may be paced in VOO or VVI mode in parallel with the IMD performing the impedance measurements in qualification test (1).

The processor 80 may control the signal generator 84 to deliver the pacing pulses during or after impedance measurement according to the qualification test parameters 83 stored in the memory 82. For example, the processor 80 may control the timing or amplitude of test pulses based on the qualification test parameters 83 which, in some examples, can specify a period of time, e.g., a window, subsequent a detected cardiac event, which may be an amplitude of an R-wave or a P-wave, or other electrogram measurement, noise, an asystolic EGM signal, or the like, in which one or more pacing pulses may be delivered. In some examples, the duration of the period may be selected as appropriate to determine the most accurate impedance values. Furthermore, by controlling the timing of pacing pulses in this manner, the IMD may avoid interference with the accuracy of impedance measurements by intrinsic cardiac signals. The processor 80 may compare the impedances measured from each of the test pulses to an impedance threshold, and evaluate the integrity of the sensing configuration, or more generally lead integrity, based upon the comparison.

In other examples, the processor 80 may also switch from the current sensing configuration to an alternative sensing configuration in response to determining that the detection of the EGM signal may have been due to a lead related condition or other integrity issue with the sensing configuration. The processor 80 may select the alternative sensing configuration from a list of available sensing configurations stored in the memory 82. In some examples, multiple sensing configurations, e.g., electrode combinations, may be tested in response to the detection of the EGM signal, and a sensing configuration that does not exhibit an integrity issue may be selected.

Additionally, the processor 80 may change the stimulation configuration if the integrity test indicates a potential issue with the stimulation configuration delivering effective therapy to the patient 14. For example, if the sensing configuration utilizes one or more electrodes also used to deliver stimulation, e.g., a pacing pulse or a shock, the processor 80 may switch to an alternative stimulation configuration that no longer includes the one or more electrodes.

In some examples, the IMD 126 continuously performs the qualification test (1) until the processor 80 determines a passing impedance measurement over a selected time interval. If the measured impedance during the selected time interval is not within a predetermined passing range, qualification test (1) is determined by the processor 80 to be a failure, and the result is optionally stored in the memory 82. If the measured impedance in qualification test (1) is within the passing range, the qualification test (1) is determined by the processor 80 to be a pass, and the result may optionally be stored in the memory 82. If the qualification test (1) is determined to be a pass, in some examples the processor 80 may cause the signal generator 84 and the sensing module 86 to initiate pacing of the heart in, for example, VVI mode, prior to initiation of qualification tests (2) or (2)-(3). For example, in some cases, the pacing is applied by the IMD to the heart at 60 bpm at a pacing output of about 5 V.

In some examples, within a selected time such as, for example, about 30 minutes to about 24 hours, following completion of qualification test (1), the processor 80 initiates qualification test (2). In qualification test (2), the processor 80 may detect sensed cardiac events from EGM signals such as, for example, R-wave, P-wave, and current of injury (COI) test criteria 85 stored within the memory 82. In some examples, the EGM test criteria 85 may include one or more R-wave or P-wave thresholds, to which the processor 80 may compare a count in an R-R or P-P interval counter. In one example, the suitable EGM thresholds may include any EGM signal in which the measurable R-R (or P-P) waves have an amplitude of at least about 5 mV at a rate of at least about 40 beats per minute (bpm). If the EGM threshold is satisfied, the qualification test (2) is completed and the processor 80 initiates the qualification test (3).

In some examples, if the initial measurement by the sensing module 86 determines that the EGM rate is not a pass and is, for example, less than 40 bpm, the processor 80 may change the EGM sensing parameters, e.g., amplitude threshold, and/or decrement the acceptable EGM rate, and the EGM test criteria may be applied until a predetermined number of waves having the threshold rate is detected and the passing EGM threshold is achieved.

In some examples, the processor 80 may perform cardiac current of injury (COI) tests according to COI test parameters 85 in the memory 82, following the completion of the EGM qualification test, or in parallel with the EGM qualification test. In some examples, the sensing module 86 may sense the heart in, for example, OVO mode, prior to initiation of the COI test parameters stored in the memory 82. Suitable COI parameters 85 in the memory 82 include, but are not limited to, determination of: the maximum amplitude of the ST segment in the electrogram of the patient, amplitude of the ST segment 80 milliseconds from the segment's beginning, the area under the wave curve (from R-wave start to the end of the ST segment), and the area under the ST segment, and combinations thereof. In some examples, the other EGM signals such as R-wave or P-wave amplitude may be evaluated over the same time period that the COI parameters are determined. If insufficient COI is detected, or if insufficient EGM amplitudes are detected, the qualification test (2) may be terminated by the processor 80.

If the processor 80 outputs that the IMD passes qualification test (2), within a selected time such as, for example, about 30 minutes to about 24 hours, in some examples the processor 80 optionally initiates qualification test (3) to monitor the pacing capture threshold (PCT) of the IMD according to the PCT/VCM (ventricular capture management) parameters 87 in the memory 82. In the PCT qualification test, the VCM module 89, the sensing module 86, and the signal generator 84 apply PCT parameters stored in the memory 82 to evaluate the energy required to cause depolarization and contraction of the heart tissue of the patient, and compares the required energy to a PCT threshold. For example, in some embodiments, the PCT threshold may be evaluated by the VCM module 89 to automatically monitor pacing thresholds at periodic intervals. Once the pacing threshold is determined, in some examples the VCM module determines a target heart output based on a predetermined safety margin and a predetermined minimum EGM amplitude.

In some examples, the processor 80 may run abbreviated VCM parameters in which the PCT capture threshold is required to be less than about 2.5 V. In some examples, the processor 80 initiates the signal generator 84 to pace the heart in VVI mode for a predetermined time period (for example, 30 seconds to 60 seconds) to evaluate PCT capture threshold. In some examples, the heart is paced in VVI mode at about 90 bpm to determine the PCT capture threshold. Such a pacing rate is likely faster than the intrinsic heart rate of the patient so that intrinsic heart beats will not interfere with pacing capture threshold testing.

If the PCT capture threshold is not achieved, the processor 80 indicates that qualification test (3) was not successful, and the result may optionally be stored in the memory 82.

At any time following the completion of implantation of the IMD 126, or if the processor 80 outputs that the IMD passes qualification test (3), within a selected time such as, for example, about 30 minutes to about 24 hours, in some examples the processor 80 optionally initiates qualification test (4) to measure clinical or patient specific physiologic metrics and compare the measured physiologic metrics to a physiologic metrics threshold to determine readiness to discharge the patient. In some examples, which are not intended to be limiting, the IMD may measure one or more patient specific or clinical physiologic metrics such as heart rate, respiration rate, activity level, thoracic impedance, blood pressure, ejection fraction, heart sounds, arrythmia burden, required percent pacing, and combinations thereof.

If any or all of the qualification tests (1)-(4) are determined to be in a range indicating that the implantation of the IMD 126 was successful, in some examples the processor 80 generates a status signal within the second predetermined time, and a result is optionally stored in the memory 82. Suitable indications for the status signal include, for example, energizing a LED, an audible alert, or sending a pass/complete signal to an optional programmer 130 or Medtronic CARELINK.

If any or all of the qualification tests (1)-(4) are determined to be in a range indicating that the implantation of the IMD 126 was successful, either before or after the patient 116 is discharged from the hospital, the IMD 126 may initiate a second device test sequence different from the initial device test sequence. For example, the initial device test sequence may include different parameters stored in the memory 82 compared to the second device test sequence. In one example, qualification tests in the initial device test sequence, which are determined based on a range of values output by the IMD 126 on the day of implantation, may include less stringent pass criteria than the qualification tests in the second device test sequence, which includes values selected for longer term monitoring of IMD performance and device capture.

The various components of the IMD 126 are coupled to a power source 90, which may include a rechargeable or non-rechargeable battery. A non-rechargeable battery may be capable of holding a charge for several years, while a rechargeable battery may be inductively charged from an external device, e.g., on a daily or weekly basis.

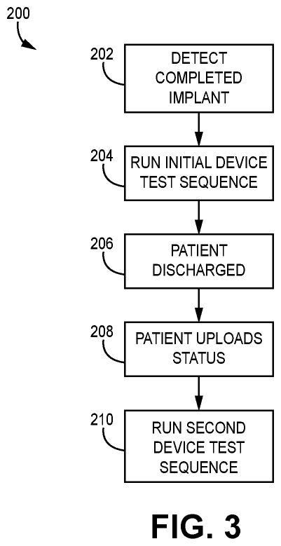

FIG. 3 is a flow diagram illustrating an embodiment of a device test sequence 200 performed by an 1 MB 126 (for example, FIG. 1) according to the present disclosure. In step 202, the 1 MB 126 detects completion of an implant procedure in which the 1 MB is implanted in a patient. In some examples, after the IMD 126 detects attachment of the lead 112, or other indication of operation of the IMD 126, to determine that implant detect is complete.

In step 204, the processor 80 automatically initiates, within a first predetermined time from detection of implant completion (e.g., about 0.5 hour to about 8 hours from detecting the implant completion), an initial device test sequence including one or more of the qualification tests (1)-(4) described herein.

In one of example of qualification test (1), the signal generator 84 (FIG. 2) paces the ventricle of the heart in VOO mode at a rate of about 80 pulses per minute, and the impedance measurement module 92 performs the qualification test (1) after each paced event to determine if the measured lead impedance is within a passing range. If the qualification test (1) is determined to be a pass, the algorithm proceeds to perform either or all of the qualification tests (2)-(4) in the device test sequence. If the measured impedance is outside the passing range, the qualification test (1) is deemed a fail, and in some examples the algorithm can re-initiate the qualification test (1) and search for a passing lead impedance value. As noted above, the algorithm may continue the qualification test (1) for a predetermined period of time such as, for example, about 30 minutes. If no passing lead impedance value is measured, in some examples the IMD 126 may provide a notification of implant failure.

If qualification test (1) is a pass, the algorithm may initiate the qualification test (2) by applying by the processor any or all of the P-wave, R-wave, or COI test criteria 85 stored in the memory 82. For example, if the measured P-wave or R-wave amplitude is greater than a predetermined threshold, the qualification test (2) is deemed a pass and the algorithm moves to qualification test (3). If the measured amplitudes do not meet the predetermined threshold, if too few P-waves or R-waves are detected, or if excessive noise is detected in the EGM signal, qualification test (2) is deemed a fail and the algorithm returns a non-qualification signal, and optionally re-initiates the qualification test (1).

Prior to, in parallel with, or after the P-wave or R-wave sensing, in some examples the algorithm may optionally measure and calculate relevant COI parameters 85 as stored in the memory 82. In various examples, as noted above, these COI parameters 85 can include the maximum amplitude of the ST segment, the amplitude of the ST segment 80 milliseconds from the segment's beginning, the area under the wave curve (from R-wave start to the end of the ST segment), the area under the ST segment, and combinations thereof. If insufficient COI is detected after an evaluation period, the algorithm outputs that qualification test (2) is a fail, and the IMD repeat qualification test (2) or returns to qualification test (1).

In qualification test (3), the processor 80 causes the VCM module 89 to perform a VCM test to determine a pacing capture threshold (PCT). In one example, if the VCM is determined at 2.5V without reference to ventricular pacing, the PCT is evaluated by the VCM module 89 and device capture is confirmed. If the PCT is outside the target range at 2.5V, the device provides a non-confirmation signal or returns to qualification test (1) or (2).

In one example, once capture confirmation is obtained, the algorithm proceeds after a 30 second interval by initiating pacing in VVI mode. If the VVI pacing protocol is successful, the qualification test (3) is deemed a pass.

Prior to, or in parallel with, any of qualification tests (1)-(3), the processor 80 may cause the sensing module 86 to perform qualification test (4) and measure clinical or patient specific physiologic metrics such as such as, for example, one or more of heart rate, respiration rate, activity level, thoracic impedance, blood pressure, ejection fraction, heart sounds, arrythmia burden, and required percent pacing. The processor 80 may compare any or all of the measured physiologic metrics against a physiologic metrics threshold to determine the potential readiness of the patient for discharge.

If the selected qualification tests (1)-(4) are successfully completed, within a second predetermined time period the IMD 126 optionally sends to the external device 130 a status signal indicating a status of at least one of the diagnostic tests in the first device test sequence. In some examples, the IMD 126 then re-runs the device test sequence and provides the status signal at least once every 3 hours over a 24 hour implant evaluation period. In some examples, the status signals are stored in the memory 82, and may be reviewed by a clinician on the external device 130 by interrogating the IMD 126.

Following discharge of the patient from the hospital in step 206, in step 208, the morning following the implant procedure, the patient 116 uploads an implant confirmation signal to the external device 130 indicating the status of the implant procedure. The upload may be reviewed by a clinician to confirm the status of the implantation of the IMD 126.

In step 210, following the upload of the implant status by the patient 116, the IMD 126 may automatically initiate a second device test sequence different from the initial device test sequence performed in step 204. As noted above, qualification tests in the initial device test sequence, which are determined based on a range of values output by the IMD 126 on the day of implantation, may include less stringent pass criteria than the qualification tests in the second device test sequence. The second device test sequence may include values selected for longer term monitoring of IMD performance and device capture.

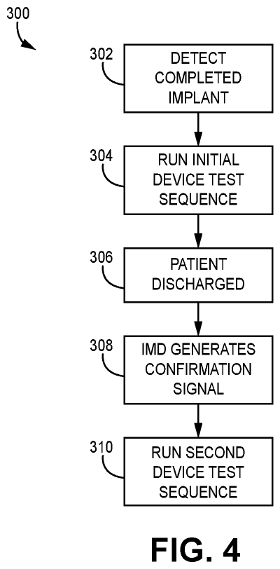

FIG. 4 is a flow diagram illustrating another embodiment of a method 300 performed by an 1 MB 126 (for example, FIG. 1) according to the present disclosure. In step 302, the 1 MB 126 detects completion of an implant procedure. In some examples, after the IMD 126 detects attachment of the lead 112, or other indication of operation of the IMD 126, to determine that implant detect is complete.

In step 304, the processor 80 automatically initiates, within a first predetermined time from detection of implant completion (e.g., within about 0.5 hour to about 8 hours from detecting the implant completion), an initial device test sequence including one or more of the qualification tests (1)-(4) described above with reference to FIG. 3.

If the selected qualification tests (1)-(4) are successfully completed, within the second predetermined time the IMD 126 optionally sends to the external device 130 a status signal indicating a status of at least one of the diagnostic tests in the first device test sequence. In some examples, the IMD 126 then re-runs the device test sequence and provides the status signal at least once every 3 hours (or any other predetermined interval of time) over a 24 hour implant evaluation period. In some examples, the status signals are stored in the memory 82, and may be reviewed by a clinician on the external device 130 by interrogating the IMD 126.

Following discharge of the patient from the hospital in step 306, in step 308 the IMD 126 automatically generates and transmits to the external device 130 an implant confirmation signal to the external device indicating the status of the implant procedure, and no manual patient uplink to the external device is required. The implant confirmation signal may be reviewed by a clinician to determine the success or failure of the implantation of the IMD 126. In various examples, the confirmation signal may be an aggregate of the values of the qualification tests (1)-(4) in the device test sequence, or a summary of the values of the qualification tests (1)-(4), or even a simple pass/complete, pass/fail, or incomplete.

In step 310, the IMD 126 may automatically initiate a second device test sequence different from the initial device test sequence performed in step 304. As noted above, the qualification test parameters for the second device test sequence may be more stringent than the qualification test parameters in the initial device test sequence, because the heart of the patient may in some cases be more stable on the days after implant than on the day of implant. The second device test sequence may include values selected for longer term monitoring of IMD performance and device capture.

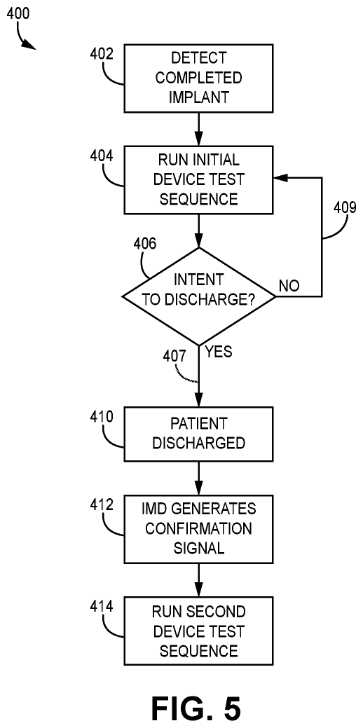

FIG. 5 is a flow diagram illustrating another embodiment of a method 400 performed by an 1 MB 126 (for example, FIG. 1) according to the present disclosure. In step 402, the 1 MB 126 detects completion of an implant procedure. In some examples, after the IMD 126 detects attachment of the lead 112, or other indication of operation of the IMD 126, to determine that implant detect is complete.

In step 404, the processor 80 automatically initiates, within a first predetermined time from detection of implant completion (e.g., within about 0.5 hour to about 8 hours from detecting the implant completion), an initial device test sequence including one or more of the qualification tests (1)-(4) described above with reference to FIGS. 3-4.

If the selected qualification tests (1)-(4) are successfully completed, within a second predetermined time the IMD 126 optionally sends to the external device 130 a status signal indicating a status of at least one of the diagnostic tests in the first device test sequence. In some examples, the IMD 126 then re-runs the device test sequence and provides the status signal at least once every 3 hours (or any other predetermined interval of time) over a 24 hour implant evaluation period. In some examples, the status signals are stored in the memory 82, and may be reviewed by a clinician on the external device 130 by interrogating the IMD 126.

In step 406, the IMD 126 determines an intention to discharge the patient from the hospital.

In one example, the IMD determines intent to discharge the patient based on an accelerometer signal from the accelerometer 91, and discharge parameters 93 stored in the memory module 82, indicating that the patient 116 is upright and out of bed following implantation of the IMD 126. In some examples, the processing circuitry 80 may be configured to identify, based on the signals from the accelerometer 91 and the discharge parameters 93, that the patient 116 is ambulatory. Either or both of the upright posture of the patient 116 and the ambulatory status of the patient 116 may be used to determine that the patient 116 is ready to be discharged from a hospital following implantation of the IMD 126. The comparison of the output signals of the accelerometer 91 to the discharge parameters 93 may be performed following the completion of the initial device test sequence performed by the IMD 126 in step 404, or in parallel with the initial device test sequence performed in step 404.

In some examples, the discharge parameters 93 stored in the memory 82 may further optionally include location information for the patient 116 or of the hospital where the IMD 126 is implanted. In some examples, the IMD 126 may detect a location of the patient 116, and determine, based on the detected location information, a discharge status of the patient, or that the patient has already been discharged from the hospital. In some examples, the IMD may sense or detect any of the physiologic parameters 95 stored in the memory 82, and the processor 80 may use these physiologic parameters to determine the discharge status of the patient, or to determine that the patient is ready for discharge.

If there is an intent to discharge the patient in step 406, in step 407 the patient status is moved to a discharge protocol in step 410. If there is no intent to discharge the patient in step 406, in step 409 the IMD 126 repeats the initial device test sequence performed in step 404, again determines the intent to discharge in step 406, and repeats these steps until the patient is discharged from the hospital in step 410.

In step 412, the IMD 126 automatically generates and transmits to the external device 130 an implant confirmation signal to the external device indicating the status of the implant procedure. The upload may be reviewed by a clinician to determine the success or failure of the implantation of the IMD 126. In various examples, the confirmation signal may be an aggregate of the values of the qualification tests (1)-(4) in the device test sequence, or a summary of the values of the qualification tests (1)-(4), or even a simple pass/complete, pass/fail, or incomplete.

In step 414, following the upload of the implant status by the patient 116, the IMD 126 may automatically initiate a second device test sequence different from the initial device test sequence performed in step 404. As noted above, the qualification test parameters for the second device test sequence may be more stringent than the qualification test parameters in the initial device test sequence, because the heart of the patient may in some cases be more stable on the days after implant than on the day of implant. The second device test sequence may include values selected for longer term monitoring of IMD performance and device capture.

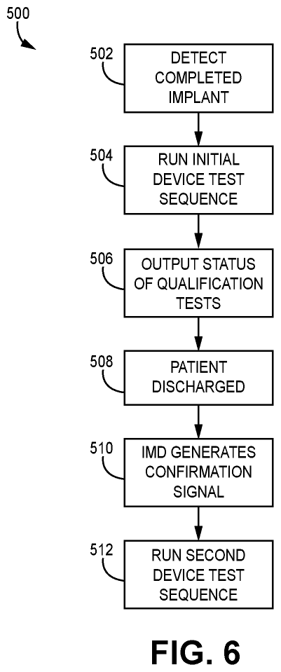

FIG. 6 is a flow diagram illustrating another embodiment of a method 500 performed by an 1 MB 126 (for example, FIG. 1) according to the present disclosure. In step 502, the 1 MB 126 detects completion of an implant procedure. In some examples, after the IMD 126 detects attachment of the lead 112, or other indication of operation of the IMD 126, to determine that implant detect is complete.

In step 504, the processor 80 automatically initiates, within a first predetermined time from detection of implant completion (e.g., about 0.5 hour to about 8 hours from detecting the implant completion), an initial device test sequence including one or more of the qualification tests (1)-(4) described herein with respect to FIGS. 3-5 above.

If the selected qualification tests (1)-(4) are successfully completed, in step 506 the IMD 126 automatically sends to the external device 130 within a second predetermined time a status signal indicating a status of at least one of the diagnostic tests in the first device test sequence. In some examples, the IMD utilizes Bluetooth low energy (BLE) advertising packets to provide the status signal to the external device 130. For example, the status signal generated by the IMD 126 may include an aggregate value indicating the success or failure of the implantation of the IMD 126, or a summary of one or more of the values of the qualification tests (1)-(4). In one example, the summary in the status signal may be limited to complete/pass, complete/fail, and incomplete.

In various examples, the IMD 126 transmits the status signals at predetermined intervals of time (e.g., at least once every 2 hours following implantation, once every 3 hours, once every 4 hours, once every 6 hours). In some examples, the confirmation signals are stored in the memory 82, and may be reviewed by a clinician on the external device 130 without necessitating by the external device 130 an interrogation of the IMD 126.

In step 510, following the discharge of the patient in step 508, the IMD 126 automatically generates and transmits to the external device 130 an implant confirmation signal to the external device indicating the status of the implant procedure. The upload may be reviewed by a clinician to determine the success or failure of the implantation of the IMD 126. In various examples, the confirmation signal may be an aggregate of the values of the qualification tests (1)-(4) in the device test sequence, or a summary of the values of the qualification tests (1)-(4), or even a simple pass/complete, pass/fail, or incomplete.

In step 512, following the upload of the implant status by the patient 116, the IMD 126 may automatically initiate a second device test sequence different from the initial device test sequence performed in step 504. For example, the initial device test sequence may include different parameters stored in the memory 82 compared to the second device test sequence. As noted above, the qualification test parameters for the second device test sequence may be more stringent than the qualification test parameters in the initial device test sequence. The heart of the patient may in some cases be more stable on the days after implant than on the day of implant, and the second device test sequence may include values selected for longer term monitoring of IMD performance and device capture.

It should be noted that the therapy system 100 may not be limited to treatment of a human patient. In alternative examples, the therapy system 100 may be implemented in non-human patients, e.g., primates, canines, equines, pigs, and felines. These other animals may undergo clinical or research therapies that my benefit from the subject matter of this disclosure.

The techniques described in this disclosure, including those attributed to the IMD 126, the programmer 130, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

The following examples are illustrative of the techniques of this disclosure.

Example 1. A method comprising initiating, by an implantable medical device (IMD), within a first predetermined time following an implantation of the IMD in a patient, a first device test sequence in which the IMD performs at least two of the following qualification tests over an evaluation period: (1) detecting an impedance for at least one electrical path that includes at least one electrode, and comparing the impedance to a first predetermined impedance threshold to determine a connection status of the IMD to the at least one electrode; (2) comparing, over an electrogram (EGM) test period, at least one EGM event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metric threshold. The method further comprises transmitting, by the IMD, to an external device, within a second predetermined time, a status signal indicating a status of at least one of the diagnostic tests in the first device test sequence.

Example 2. The method of example 1, wherein the IMD comprises an accelerometer, and the first diagnostic test sequence further comprises a qualification test (5) comprising determining, with the accelerometer, at least one of body posture or ambulation to determine a discharge status of the patient.

Example 3. The method of example 1 or 2, further comprising determining, by the IMD, a location of the patient, and determining, by the IMD, a discharge status of the patient based on the location of the patient.

Example 4. The method of any one or more examples 1 to 3, wherein the IMD initiates the first device test sequence prior to an expected discharge time of the patient.

Example 5. The method of any one or more of examples 1 to 4, wherein the IMD transmits the status signal at least once every 3 hours following the implantation.

Example 6. The method of any one or more of examples 1 to 5, wherein the IMD initiates the first device test sequence no later than about 1 hour following the implantation.

Example 7. The method of any one or more of examples 1 to 6, wherein the evaluation period is about 24 hours following the implantation.

Example 8. The method of any one or more of examples 1 to 7, wherein the first device test sequence includes diagnostic tests (1)-(2).

Example 9. The method of any one or more of examples 1 to 7, wherein the first device test sequence includes diagnostic tests (1)-(4).

Example 10. The method of example 2, wherein the first device test sequence includes diagnostic tests (2)-(5).

Example 11. The method of any one or more of examples 1 to 7, wherein the first device test sequence includes diagnostic test (4).

Example 12. The method of any one or more of examples 1 to 11, wherein the second predetermined time is about every 2 hours following completion of the device test sequence.

Example 13. The method of example 2, wherein the second predetermined time is about every 2 hours following completion of the device test sequence.

Example 14. The method of any one or more of examples 1 to 13, wherein the status signal comprises an aggregate of a status of at least two of the diagnostic tests (1)-(4).

Example 15. The method of any one or more of examples 1 to 14, wherein the status signal comprises a summary of a status of at least two of the diagnostic tests (1)-(4).

Example 16. The method of example 15, wherein the status signal is chosen from complete/pass, complete/fail, and incomplete.

Example 17. The method of any one or more of examples 1 to 16, wherein the status signal comprises a comparison of results of any or all of the diagnostic tests (1)-(4), and wherein the comparison is provided in a report to a clinician.

Example 18. The method of any one or more of examples 1 to 17, wherein the external device comprises one or more of a mobile phone, a tablet, or a device programmer.

Example 19. The method of example 18, wherein the mobile phone is at least one of a mobile phone associated with the patent or a clinician.

Example 20. The method of any one or more of examples 1 to 19, wherein within a third predetermined time following the transmission of the status signal, the IMD transmits an implant confirmation signal to the external device.

Example 21. The method of example 20, wherein the third predetermined time is less than 24 hours.

Example 22. The method of any one or more of examples 1 to 21, wherein following transmitting of the status signal, the method comprise performing, by the IMD, a second device test sequence different from the first device test sequence.

Example 23. The method of example 22, wherein the second device test sequence comprises at least one of the following diagnostic tests: (1) determining a second pacing capture threshold (PCT) of the IMD, wherein the second PCT is different from the first PCT; 2) comparing, over an EGM test period, at least one EGM event of the patient against a second predetermined EGM event threshold different from the first EGM event threshold; (3) comparing the impedance to a second predetermined impedance threshold, different from the first predetermined impedance threshold; and (4) comparing clinical or patient-specific physiologic metrics to a second predetermined physiologic metrics threshold different from the first predetermined physiologic metrics threshold. The method further comprises transmitting by the IMD, to the external device, a second status signal indicating a status of at least one of the diagnostic tests in the second device test sequence.

Example 24. A method, comprises initiating, by an implantable medical device (IMD), within a first predetermined time following an implantation of the IMD in a patient, a first device test sequence in which the IMD performs at least one of the following qualification tests over an evaluation period: (1) detecting an impedance for at least one electrical path that includes at least one electrode, and comparing the impedance to a first predetermined impedance threshold to determine a connection status of the IMD to the at least one electrode; (2) comparing, over an EGM test period, at least one electrogram (EGM) event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metrics threshold. The method further comprises transmitting by the IMD, to an external device, within a second predetermined time a status signal indicating a status of at least one of the diagnostic tests in the first device test sequence.

Example 25. The method of example 24, wherein the transmitting by the IMD of the status signal occurs at least once every 3 hours following the implantation.

Example 26. The method of example 24 or 25, wherein the evaluation period is about 24 hours following the implantation.

Example 27. The method of any one or more of examples 24 to 26, wherein within a third predetermined time following the status signal, the IMD transmits an implant confirmation signal to the external device.

Example 28. The method of example 27, wherein the third predetermined time is less than 24 hours.

Example 29. The method of any one or more of examples 24 to 28, wherein following transmitting of the status signal, the IMD performs a second device test sequence different from the first device test sequence.

Example 30. A method comprises initiating, by an implantable medical device (IMD) comprising an accelerometer, within a first predetermined time following an implantation of the IMD in a patient, a first device test sequence in which the IMD performs at least two of the following qualification tests over an evaluation period: (1) detecting an impedance, and comparing the impedance to a first predetermined impedance threshold to determine a connection status of the IMD to at least one electrode; (2) comparing, over an EGM test period, at least one electrogram (EGM) event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metrics threshold. The method further comprises transmitting by the IMD, to an external device, within a second predetermined time period, a status signal indicating a status of at least one of the diagnostic tests in the first device test sequence, and determining, by the IMD, a discharge status of the patient by at least one of: determining, by the accelerometer in the IMD, at least one of a body posture or an ambulation of the patient, and comparing the at least one of body posture or ambulation of the patient against a threshold for discharge; or determining, by the IMD, a patient location.

Example 31. The method of example 30, comprising periodically performing, by the IMD, at least some of the qualification tests (1)-(4) in the first device test sequence until the IMD confirms the patient is discharged.

Example 32. The method of example 31, wherein the transmitting by the IMD of the status signal occurs at least once every 3 hours following the implantation.

Example 33. The method of any one or more of examples 30 to 32, wherein the evaluation period is about 24 hours following the implantation.

Example 34. The method of any one or more of examples 30 to 33, wherein within a third predetermined time period following the first confirmation signal, the IMD transmits an implant confirmation signal to the external device.

Example 35. The method of example 34, wherein the third predetermined time is less than 24 hours.

Example 36. The method of any one or more of examples 30 to 35, wherein following transmitting of the status signal, the IMD performs a second device test sequence different from the first device test sequence.

Example 37. A method comprises initiating, by an implantable medical device (IMD), within a first predetermined time following an implantation of the IMD in a patient, a first device test sequence in which the IMD performs at least one of the following qualification tests over an evaluation period: (1) detecting an impedance for at least one electrical path that includes at least one electrode, and comparing the impedance to a first predetermined impedance threshold to determine a connection status of the IMD to the at least one electrode; (2) comparing, over an EGM test period, at least one electrogram (EGM) event of the patient against a first predetermined EGM event threshold; (3) determining a first pacing capture threshold (PCT) of the IMD; and (4) detecting at least one clinical or patient-specific physiologic metric, and comparing the physiologic metric to a first predetermined physiologic metrics threshold. The method further comprises transmitting by the IMD, to an external device, within a second predetermined time period a status signal indicating a status of at least one of the diagnostic tests in the first device test sequence, wherein the first status signal is chosen from complete/pass, complete/fail, and incomplete.

Example 38. The method of example 37, wherein the transmitting by the IMD to the external device comprises a Bluetooth low energy (BLE) advertising packet.

Example 39. The method of example 37 or 38, wherein the transmitting by the IMD of the status signal occurs at least once every 3 hours following the implantation.

Example 40. The method of any one or more of examples 37 to 39, wherein the evaluation period is about 24 hours following the implantation.

Example 41. The method of any one or more of examples 37 to 40, wherein within a third predetermined time following the first confirmation signal, the IMD transmits an implant confirmation signal to the external device.

Example 42. The method of example 41, wherein the third predetermined time is less than 24 hours.

Example 43. The method of any one or more of examples 37 to 42, wherein following transmitting of the status signal, the IMD performs a second device test sequence different from the first device test sequence.

Example 44. A system comprises at least one implantable medical lead comprising one or more electrodes, and an implantable medical device (IMD) coupled to the at least one lead, wherein the at least one lead senses a cardiac electrogram (EMG) signal of a patient via the electrodes. The IMD comprises a processor that causes the IMD to initiate, following implantation of the IMD, a device test sequence in which the IMD performs any of the methods of the previous claims.

Various examples have been described. These and other examples are within the scope of the following claims.

What is claimed is:

1. An implantable medical device (IMD) configured to be coupled to at least one implantable medical lead, wherein the IMD comprises:
a telemetry circuitry configured for wireless communication with an external device;
sensing circuitry configured to sense an electrogram (EMG) signal of a patient via the implantable medical lead;
impedance measurement circuitry to measure impedance via the implantable medical lead;
a signal generator configured deliver cardiac pacing via the implantable medical lead; and
a processor configured, within a first predetermined time following an implantation of the IMD in a patient, to initiate a device test sequence comprising at least two of the following qualification tests over an evaluation period:
(1) detecting an impedance for at least one electrical path that includes at least one electrode of the implantable medical lead, and comparing the impedance to a first predetermined impedance threshold to determine a connection status of the IMD to the at least one electrode;
(2) comparing, over an EGM test period, at least one EGM event of the patient against a first predetermined EGM event threshold;
(3) controlling the signal generator and the sensing circuitry to determine a first pacing capture threshold (PCT); and
(4) detecting at least one clinical or patient-specific physiologic metric, and comparing the physiologic metric to a first predetermined physiologic metric threshold; and
transmit, via the telemetry circuitry, to the external device, within a second predetermined time, a status signal indicating a status of at least one of the diagnostic tests in the first device test sequence.

2. The IMD of claim 1, wherein the processor is configured to:
determine a discharge status of the patient; and
initiate the device test sequence based on the discharge status.

3. The IMD of claim 2, further comprising an accelerometer, wherein the processor is configured to:
determine at least one of body posture or ambulation; and
determine the discharge status of the patient based on the at least one of body posture or ambulation.

4. The IMD of claim 2, wherein the processor is further configured to:
determine a location of the patient; and
determining the discharge status of the patient based on the location of the patient.

5. The IMD of claim 1, wherein the processor is configured to initiate the first device test sequence prior to an expected discharge time of the patient.

6. The IMD of claim 1, wherein the processor is configured to transmit the status signal at least once every 3 hours following the implantation.

7. The IMD of claim 1, wherein the processor is configured to initiate the first device test sequence no later than about 1 hour following the implantation.

8. The IMD of claim 1, wherein the evaluation period is about 24 hours following the implantation.

9. The IMD of claim 1, wherein the first device test sequence includes diagnostic tests (1)-(2).

10. The IMD of claim 1, wherein the first device test sequence includes diagnostic tests (1)-(4).

11. The IMD of claim 1, wherein the first device test sequence includes diagnostic test (4).

12. The IMD of claim 1, wherein the second predetermined time is about every 2 hours following completion of the device test sequence.

13. The IMD of claim 1, wherein the status signal comprises at least one of an aggregate or a summary of a status of at least two of the diagnostic tests (1)-(4).

14. The IMD of claim 13, wherein the status signal is chosen from complete/pass, complete/fail, and incomplete.

15. The IMD of claim 1, wherein the processor is configured to, within a third predetermined time following the transmission of the status signal, transmit an implant confirmation signal to the external device.

16. The IMD of claim 15, wherein the third predetermined time is less than 24 hours.

17. The IMD of claim 1, wherein following transmitting of the status signal, the processor is configured to perform a second device test sequence different from the first device test sequence.

18. The IMD of claim 17, wherein the second device test sequence comprises at least one of the following diagnostic tests:
(1) determining a second PCT, wherein the second PCT is different from the first PCT; and
(2) comparing, over an EGM test period, at least one EGM event of the patient against a second predetermined EGM event threshold different from the first EGM event threshold;
(3) comparing the impedance to a second predetermined impedance threshold, different from the first predetermined impedance threshold; and
(4) comparing clinical or patient-specific physiologic metrics to a second predetermined physiologic metrics threshold different from the first predetermined physiologic metrics threshold,
wherein the processor is configured to transmit, to the external device, a second status signal indicating a status of at least one of the diagnostic tests in the second device test sequence.

19. A method, comprising:
initiating, by an implantable medical device (IMD), within a first predetermined time following an implantation of the IMD in a patient, a first device test sequence in which the IMD performs at least two of the following qualification tests over an evaluation period:
(1) detecting an impedance for at least one electrical path that includes at least one electrode, and comparing the impedance to a first predetermined impedance threshold to determine a connection status of the IMD to the at least one electrode;
(2) comparing, over an electrogram (EGM) test period, at least one EGM event of the patient against a first predetermined EGM event threshold;
(3) determining a first pacing capture threshold (PCT) of the IMD; and
(4) detecting at least one clinical or patient-specific physiologic metric, and comparing the physiologic metric to a first predetermined physiologic metric threshold; and
transmitting, by the IMD, to an external device, within a second predetermined time, a status signal indicating a status of at least one of the diagnostic tests in the first device test sequence.

20. The method of claim 19, further comprising:
determining a discharge status of the patient based on at least one of a location, ambulation or posture of the patient; and
initiate the device test sequence based on the discharge status.
